(19) United States
(12) Patent Application Publication
(10) Pub. No.: US 2024/0010646 A1
(43) Pub. Date: Jan. 11, 2024

(54) Urea Co-Crystal of Apixaban, and Preparation Method Therefor

(71) Applicant: CHENGDU EASTON BIOPHARMACEUTICALS CO., LTD., Sichuan (CN)

(72) Inventors: Xueyao Zeng, Sichuan (CN); Huike Gu, Sichuan (CN); Cui Huang, Sichuan (CN); Hong Chen, Sichuan (CN); Ying Wang, Sichuan (CN)

(21) Appl. No.: 18/004,276

(22) PCT Filed: Dec. 24, 2021
(86) PCT No.: PCT/CN2021/141012

§ 371 Date: Jan. 4, 2023

(30) Foreign Application Priority Data
Jun. 17, 2021 (CN) 202110669932.5

(51) Int. Cl. C07D 471/04; C07C 275/02

(52) U.S. Cl. CPC C07D 471/04; C07C 275/02; C07B 2200/13

(57) ABSTRACT

A urea co-crystal form A of apixaban, and a preparation method therefor. The urea co-crystal form A has high physical and chemical stability, crystal form stability and drug forming stability, better solubility, and higher bioavailability. The preparation process is good in repeatability, high in yield, green and environment-friendly, and easy to operate, facilitates large-scale production, and allows for preparation of products in different particle size ranges by means of adjustment of parameters, thereby meeting different requirements for formulations.

### CROSS-REFERENCE

This application is a 371 U.S. national phase of PCT/CN2021/141012, filed Dec. 24, 2021, which claims priority from CN 202110669932.5, filed Jun. 17, 2021, both which are incorporated by reference in its entirety.

### FIELD OF THE INVENTION

The present invention belongs to the field of drug crystalline form, and particularly relates to a urea co-crystal form A of apixaban and a preparation method thereof.

### BACKGROUND OF THE INVENTION

Apixaban (trade name Eliquis) is a novel oral direct factor Xa inhibitor developed jointly by Bristol-Myers Squibb and Pfizer, with the chemical formula of 1-(4-methoxyphenyl)-7-oxo-6-(4-(2-oxopiperidin-1-yl)phenyl)-4,5,6,7-tetrahydr o-1H-pyrazolo[3,4-c]pyridine-3-carboxamide. It acts directly on blood coagulation factor Xa, and is used to treat venous thrombotic diseases including deep venous thrombosis (DVT) and pulmonary embolism (PE). In May 2011, the European Union approved the marketing of apixaban (trade name Eliquis), an oral direct inhibitor of factor Xa, for use in adult patients undergoing elective hip or knee replacement surgery to prevent venous thrombembolic events (VIE). On Nov. 20, 2012, the European Commission approved Eliquis (apixaban) for the prevention of apoplexy and systemic embolism in adult patients with non-valvular atrial fibrillation (NVAF) having one or more risk factors. On Apr. 12, 2013, the launch of apixaban in China was officially announced. The structure of apixaban is shown below by Formula (I):

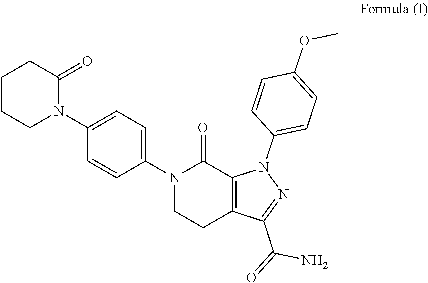 [SMILES: COc1ccc(-n2nc(C(N)=O)c3c2C(=O)N(c2ccc(N4CCCCC4=O)cc2)CC3)cc1]

Apixaban is almost insoluble in water, and has the disadvantages of slow dissolution rate, low in vitro dissolution, and low bioavailability, which have certain influence on the absorption of the medicament. Therefore, it is particularly important to seek methods to improve the dissolution of apixaban and enhance its solubility. To solve this problem, patents CN102908324, CN103830199 and CN102770126 provide other new crystalline forms of apixaban, but these new crystalline forms have problems in industrial production, such as long time-consuming, high energy consumption, low production efficiency and low yield of finished products.

Co-crystal is formed by combining an active pharmaceutical ingredient (API) molecule with a co-crystal former (CCF) such as other physiologically acceptable acid, base, salt, and non-ionic compound molecule in the same lattice via non-covalent bond such as hydrogen bond. The greatest advantage of medicament co-crystal is that it can change various physicochemical properties of the medicament without changing the covalent structure of the medicament, and the physicochemical properties of the medicament are changed in different directions and different degrees when the ligands involved in the formation of co-crystal are different, thus effectively improving the crystalline properties, physical and chemical properties, and drug efficacy of the medicament per se, and providing more options for the development of pharmaceutical solid preparation.

Patent CN106986868 held by HEC Pharm Co., Ltd. discloses four kinds of co-crystals, which are apixaban/oxalic acid, apixaban/isonicotine, apixaban/3-aminopyridine and apixaban/urea. However, among them, except for urea, the other three are not excipients approved by FDA, and have different degrees of toxicity, so there may be many regulatory restrictions on the actual use of the medicament. Meanwhile, the preparation solvent used for the disclosed urea co-crystal is trifluoroethanol, which is a non-conventional solvent and has a certain degree of toxicity, and thus it is not suitable for scale-up production, and the issue of residual solvent toxicity of the obtained product also needs to be considered.

Therefore, in order to improve the solubility of apixaban, enhance its bioavailability, and ensure the quality, safety, and efficacy of the medicament product, there is still need to develop an apixaban co-crystal form with low toxicity, good stability, and clear structure in this field.

### SUMMARY OF THE INVENTION

The present invention aims to provide a urea co-crystal form A of apixaban and a preparation method thereof, and its material basis is determined by single crystal diffraction. The resultant urea co-crystal has good stability, low toxicity, facilitates the processing of preparation, and has better solubility and higher bioavailability, and the preparation process thereof has good reproducibility and high yield, is green and environmentally friendly, and is easy to be operated and has high plasticity, and can prepare and obtain products in various particle size ranges by adjusting parameters.

The present invention provides a urea co-crystal A of the compound apixaban shown in formula (I), and the ratio of apixaban to urea in co-crystal A is 1:2,

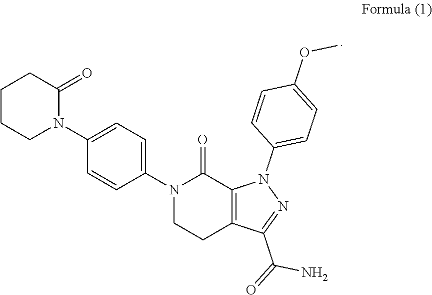 [SMILES: COc1ccc(-n2nc(C(N)=O)c3c2C(=O)N(c2ccc(N4CCCCC4=O)cc2)CC3)cc1]

Further, an X-ray powder diffraction pattern of co-crystal A has characteristic peaks at 2θ angles of 7.00±0.2°, 10.76±0.2°, 11.60±0.2°, 19.18±0.2°, 20.00±0.2°, 22.94±0.2°, 23.78±0.2° and 28.08±0.2°.

Further, the X-ray powder diffraction pattern of co-crystal A has characteristic peaks at 2θ angles of 7.00±0.2°, 10.76±0.2°, 11.60±0.2°, 12.52±0.2°, 19.18±0.2°, 22.94±0.2°, 23.78±0.2°, 25.16±0.2°, and 28.08±0.2°.

Further, the X-ray powder diffraction pattern of co-crystal A has characteristic peaks at 2θ angles of 7.00±0.2°, 10.76±0.2°, 11.60±0.2°, 12.52±0.2°, 13.96±0.2°, 16.72±0.2°, 19.18±0.2°, 20.00±0.2°, 21.18±0.2°, 22.94±0.2°, 23.78±0.2°, 25.16±0.2°, 26.88±0.2°, 28.08±0.2° and 30.20±0.2°.

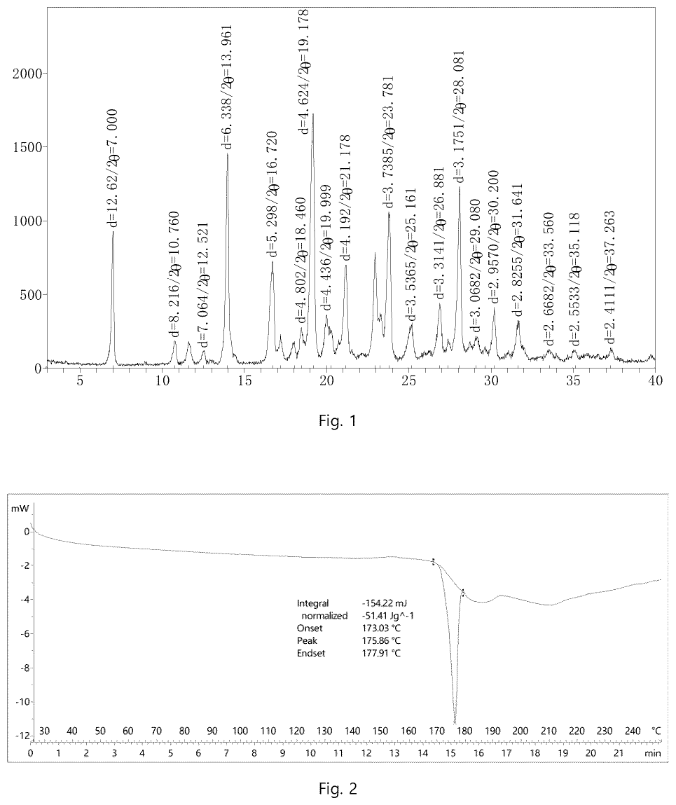

Further, the co-crystal A has an X-ray powder diffraction pattern substantially shown in FIG. 1.

Further, a DSC thermogram of the co-crystal A has an endothermic peak at 176±5° C.

Further, the DSC thermogram of the co-crystal A is substantially shown in FIG. 2.

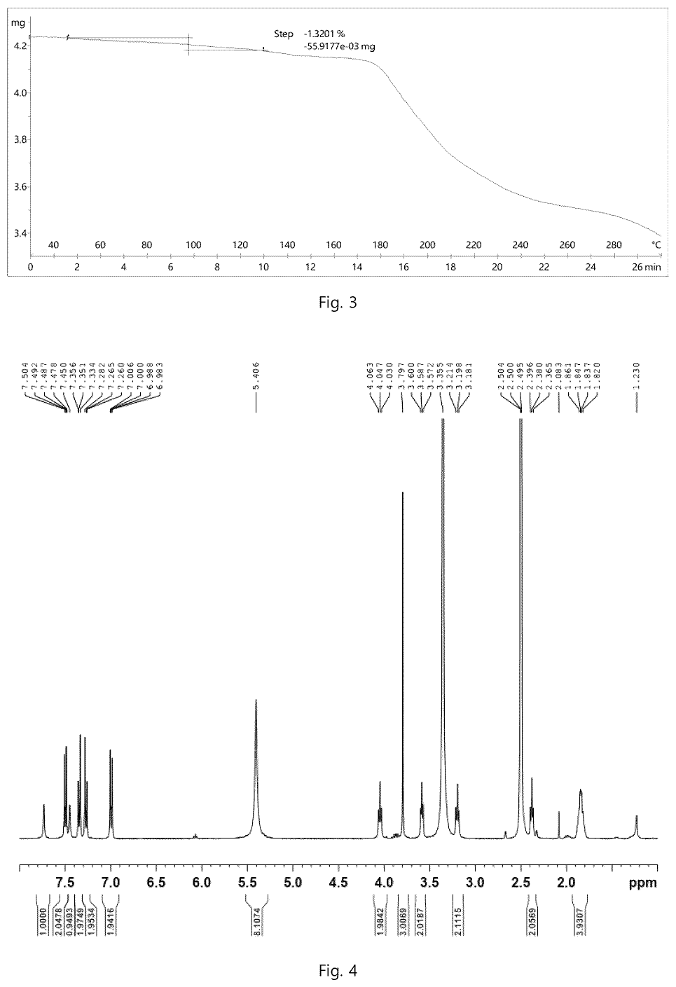

Further, a TGA diagram of the co-crystal A is substantially shown in FIG. 3. Further, a NMR spectrum of the co-crystal A is substantially shown in FIG. 4. The present invention also provides a preparation method of the urea co-crystal A of the compound shown in the above formula (I), comprising:
(1) adding the compound apixaban shown in formula (I) and a certain equivalent of urea into ethanol or a mixed solvent of ethanol and other solvents, dissolving at reflux under elevated temperature, and then cooling down to room temperature for crystallization for 5-24 h, wherein said other solvents are selected from ketones and esters;
(2) filtrating by suction and collecting the obtained solid, drying the same to yield the urea co-crystal A of apixaban.

Further, a molar ratio of apixaban to urea in step (1) is 1:4 to 1:12, preferably 1:6 to 1:10.

Further, a mass to volume ratio of apixaban to the solvent in step (1) is 1:10 to 1:30 (g/ml).

Further, said other solvents in step (1) are selected from acetone, butanone, ethyl acetate, methyl acetate or isopropyl acetate.

The present invention further provides a pharmaceutical composition of a urea co-crystal form A of apixaban, comprising the urea co-crystal form A of the compound shown in formula (I) and a pharmaceutically acceptable excipient.

The present invention also provides use of a urea co-crystal form A of apixaban, a pharmaceutical composition of a urea co-crystal form A of apixaban in preparing a medicament for diseases related to venous thrombosis.

The beneficial effects brought by the present invention include:
1. The obtained urea co-crystal form A of apixaban has relatively high physicochemical stability, crystalline form stability and medicament-forming stability, better solubility, and higher bioavailability.
2. The preparation process thereof has good reproducibility, high yield, and the solvents used thereby are all Class III solvents, and thus the preparation process is green and environmentally friendly, easy to be operated, convenient for recycling, and easy to achieve scale-up production. Meanwhile, the process has high plasticity, and can prepare and obtain products of various particle size ranges by adjusting parameters, thereby meeting different needs of preparations.

### DETAILED DESCRIPTION OF THE DRAWINGS

FIG. 1 is the XRD pattern of the urea co-crystal form A of apixaban.

FIG. 2 is the DSC thermogram of the urea co-crystal form A of apixaban.

FIG. 3 is the TGA diagram of the urea co-crystal form A of apixaban.

FIG. 4 is the ¹H-NMR spectrum of the urea co-crystal form A of apixaban.

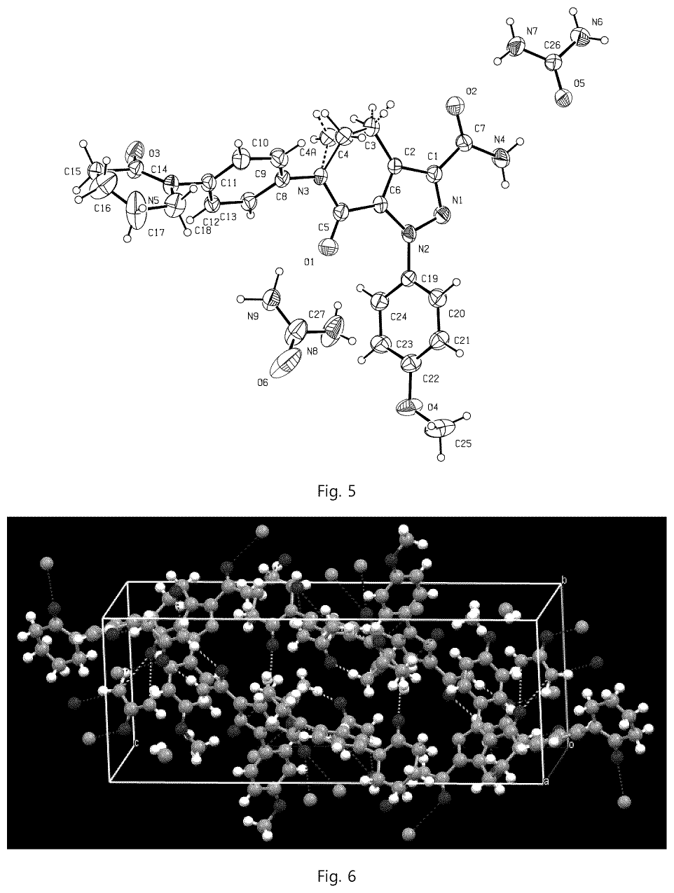

FIG. 5 is the molecular structure diagram obtained by single crystal analysis of the urea co-crystal form A of apixaban.

FIG. 6 is the single crystal cell diagram of the urea co-crystal form A of apixaban.

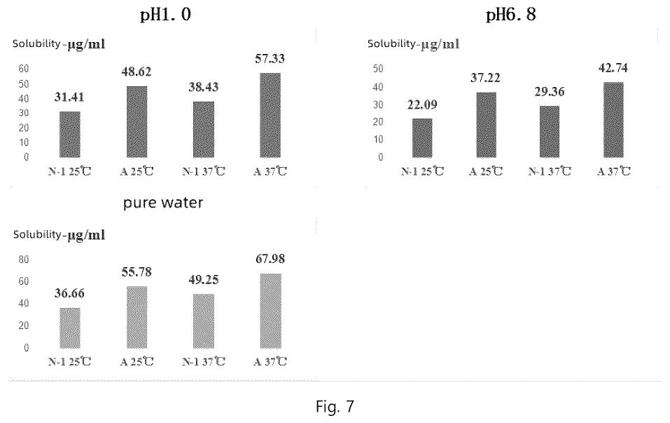

FIG. 7 is the comparison chart of the solubilities between the urea co-crystal form A of apixaban and the pharmaceutical crystalline form N−1.

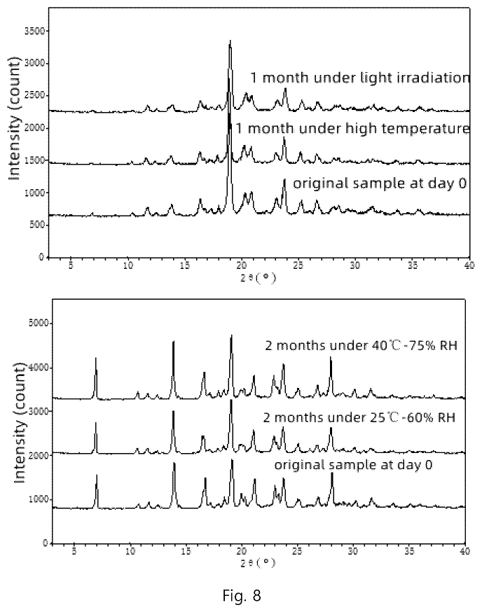

FIG. 8 is the graph of crystalline form results from the stability studies on the urea co-crystal form A of apixaban.

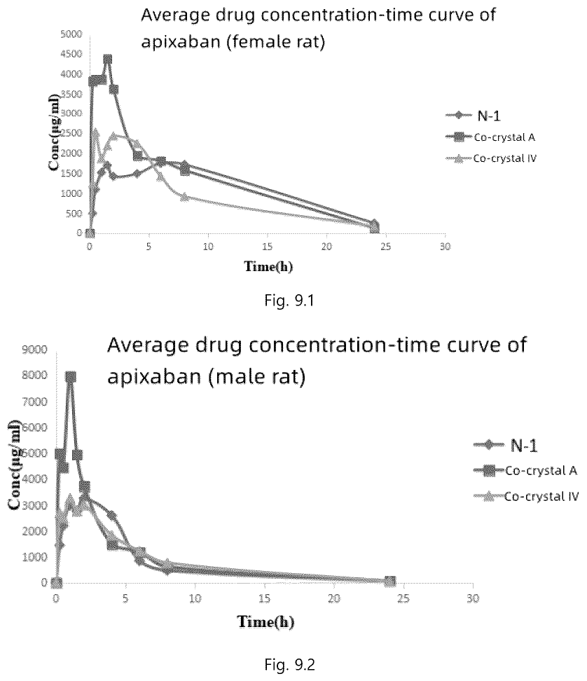

FIG. 9.1 is the mean drug concentration-time curve of the urea co-crystal form A of apixaban in female murine.

FIG. 9.2 is the mean drug concentration-time curve of the urea co-crystal form A of apixaban in male murine.

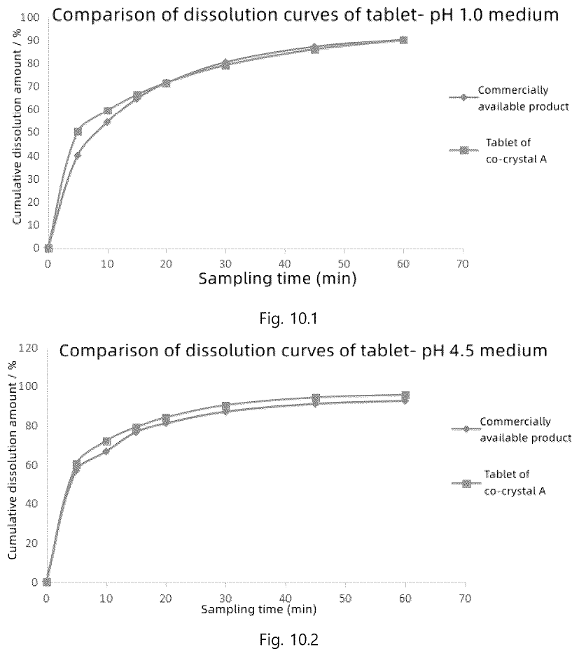

FIG. 10.1 is the comparison of the dissolution curves between the tablet prepared with the urea co-crystal form A obtained in Example 1 and commercially available product in a medium of pH 1.0.

FIG. 10.2 is the comparison of the dissolution curves between the tablet prepared with the urea co-crystal form A obtained in Example 1 and commercially available product in a medium of pH 4.5.

### EMBODIMENTS OF THE INVENTION

Hereinafter, the present invention is further described in details by referring to examples, but it is not a limitation to the present invention. Any equivalent substitution in the art made in accordance with the disclosures of the present invention falls within the protection scope of the present invention.

The abbreviations used in the present application are explained as follows:

XRD: X-ray Powder Diffraction

The data of the X-ray powder diffraction (XRD) measurement described in the present application was collected with DX-2700B powder diffractometer (Haoyuan Instrument, Dandong, Liaoning), with the specific parameters shown in the table below:

| | Reflection Parameters |
|---|---|
| X-ray reflection parameters | Cu, Kα |
| | Kα1: 1.540598; Kα2: 1.544426 |
| | Intensity ratio of Kα2/Kα1: 0.50 |
| Voltage | 40 kV |
| Current | 30 mA |
| Scan range (2θ°) | 3.0 to 40.0 degrees |
| Bragg angle (2θ°) | 0.020 degrees |

DSC: Differential Scanning Calorimeter

The data of the differential scanning calorimetry (DSC) measurement described in the present application was collected with METTLER TOLEDO model DSC-1, with a heating rate of 10° C./min, a temperature range of 25-250° C., and a nitrogen purge rate of 60 mL/min during the test.

TGA: Thermogravimetric Analyzer

The data of the thermogravimetric analysis (TGA) measurement described in the present application was collected with METTLER TOLEDO model TGA-2, with a heating rate of 10° C./min, a temperature range of 30-300° C., and a nitrogen purge rate of 20 mL/min during the test.

LC/MS/MS Biological Sample Analysis

The LC/MS/MS biological sample analysis described in the present application refers to the analysis of biological sample performed by using liquid chromatography-mass spectrometry, which has high sensitivity and high selectivity and wide applicability for analysis of mixtures, and is capable of rapid and reliable quantitative or qualitative analysis of trace compound in complex biological matrix. The liquid chromatography-mass spectrometer (mass spectrometer) involved in the present invention is AB Sciex Triple Quad 4500.

X-Ray Single Crystal Diffractometer

The measurement of the single crystal diffraction data described in the present application was collected with Rigaku XtaL AB-PRO single crystal X-ray diffractometer, with the specific parameters shown in the table below:

| Radiation | MoKα(λ = 0.71073) |
|---|---|
| 2θ range for data collection/° | 5.774 to 52.744 |
| Index ranges | −16 ≤ h ≤ 15, −10 ≤ k ≤ 11, −29 ≤ |
| | l ≤ 32 |
| Reflections collected | 15245 |
| Independent reflections | 6274[Rᵢₙₜ = 0.0659, R_{sigma} = 0.1384] |
| Data/restraints/parameters | 6274/1/394 |
| Goodness-of-fit on F² | 1.038 |
| Final R indexes [I >= 2σ (I)] | R₁ = 0.1177, wR₂ = 0.3198 |
| Final R indexes [all data] | R₁ = 0.2541, wR₂ = 0.4131 |
| Largest diff. peak/hole/e Å⁻³ | 1.45/−0.30 |

### EXAMPLES

### Example 1: Preparation of the Urea Co-Crystal A

2.3 g of Apixaban and 1.8 g of urea (6.0 eq) were weighed and added into 46.0 ml of a mixed solvent of ethyl acetate: ethanol (4:3), dissolved at reflux under elevated temperature, and then cooled down to room temperature and stirred for 18 h for crystallization. The obtained solid was collected and dried to obtain 2.75 g of nearly white urea co-crystal A of apixaban with a yield of 94.8% and a purity of 99.93%. The XRD pattern was shown in FIG. 1, the DSC thermogram was shown in FIG. 2, the TGA diagram was shown in FIG. 3, and the nuclear magnetic ¹H-NMR spectrum was shown in FIG. 4. The report of “find peaks” for characteristic peak was shown in the table below:

| 2 theta/° | d-interval/Å | area/% |
|---|---|---|
| 7.00 | 12.62 | 30.6 |
| 10.76 | 8.22 | 7.0 |
| 11.60 | 7.62 | 7.8 |
| 12.52 | 7.06 | 4.4 |
| 13.96 | 6.34 | 62.8 |
| 16.72 | 5.30 | 45.1 |
| 17.20 | 5.15 | 13.5 |
| 18.04 | 4.91 | 8.1 |
| 18.46 | 4.80 | 20.7 |
| 19.18 | 4.62 | 100.0 |
| 20.00 | 4.44 | 29.3 |
| 20.28 | 4.38 | 27.5 |
| 21.18 | 4.19 | 47.3 |
| 22.94 | 3.87 | 48.6 |
| 23.26 | 3.82 | 45.6 |
| 23.78 | 3.74 | 54.9 |
| 25.16 | 3.54 | 16.4 |
| 26.29 | 3.39 | 1.5 |
| 26.88 | 3.31 | 16.0 |
| 27.38 | 3.25 | 3.5 |
| 28.08 | 3.18 | 49.7 |
| 28.72 | 3.11 | 3.0 |
| 29.08 | 3.07 | 7.8 |
| 29.64 | 3.01 | 4.5 |
| 30.20 | 2.96 | 15.4 |
| 31.64 | 2.83 | 18.6 |
| 33.56 | 2.67 | 6.0 |
| 35.12 | 2.55 | 3.8 |
| 36.50 | 2.46 | 0.5 |
| 37.26 | 2.41 | 5.0 |

### Example 2: Preparation of the Urea Co-Crystal A

2.3 g of Apixaban and 2.4 g of urea (8.0 eq) were weighed and added into 35.0 ml of a mixed solvent of methyl acetate: ethanol (4:3), dissolved at reflux under elevated temperature, and then cooled down to room temperature and stirred for 16 h for crystallization. The obtained solid was collected and dried to obtain 2.76 g of nearly white urea co-crystal A of apixaban with a yield of 95.2% and a purity of 99.91%. The XRD spectrum was consistent with that in FIG. 1.

### Example 3: Preparation of the Urea Co-Crystal A

2.3 g of Apixaban and 3.0 g of urea (10.0 eq) were weighed and added into 58.0 ml of a mixed solvent of isopropyl acetate: ethanol (4:3), dissolved under elevated temperature, and then cooled down to room temperature and stirred for 24 h for crystallization. The obtained solid was collected and dried to obtain 2.80 g of nearly white urea co-crystal A of apixaban with a yield of 96.5% and a purity of 99.93%. The XRD spectrum was consistent with that in FIG. 1.

### Example 4: Preparation of the Urea Co-Crystal A

2.3 g of Apixaban and 3.6 g of urea (12.0 eq) were weighed and added into 69.0 ml of a mixed solvent of acetone: ethanol (4:3), dissolved at reflux under elevated temperature, and then cooled down to room temperature and stirred for 18 h for crystallization. The obtained solid was collected and dried to obtained 2.82 g of nearly white urea co-crystal A of apixaban with a yield of 97.2% and a purity of 99.92%. The XRD spectrum was consistent with that in FIG. 1.

### Example 5: Preparation of the Urea Co-Crystal A

2.3 g of Apixaban and 2.4 g of urea (8.0 eq) were weighed and added into 35.0 ml of a mixed solvent of acetone: ethanol (4:3), dissolved at reflux under elevated temperature, and then cooled down to room temperature and stirred for 5 h for crystallization. The obtained solid was collected and dried to obtain 2.79 g of nearly white urea co-crystal A of apixaban with a yield of 96.2% and a purity of 99.93%. The XRD spectrum was consistent with that in FIG. 1.

### Example 6: Preparation of the Urea Co-Crystal A

2.3 g of Apixaban and 1.2 g of urea (4.0 eq) were weighed and added into 23.0 ml of a mixed solvent of butanone: ethanol (4:3), dissolved at reflux under elevated temperature, and then cooled down to room temperature and stirred for 18 h for crystallization. The obtained solid was collected and dried to obtain 2.55 g of nearly white urea co-crystal A of apixaban with a yield of 87.9% and a purity of 99.91%. The XRD spectrum was consistent with that in FIG. 1.

### Example 7: Preparation of the Urea Co-Crystal A

2.3 g of Apixaban and 2.4 g of urea (8.0 eq) were weighed and added into 35.0 ml of a mixed solvent of acetone: ethanol (1:3), dissolved at reflux under elevated temperature, and then cooled down to room temperature and stirred for 18 h for crystallization. The obtained solid was collected and dried to obtain 2.85 g of nearly white urea co-crystal A of apixaban with a yield of 98.28% and a purity of 99.92%. The XRD spectrum was consistent with that in FIG. 1.

### Example 8: Growth of the Single Crystal of the Urea Co-Crystal a and Single-Crystal Diffraction

The inventors directly obtained the single crystal sample with large particle size and regular shape by developing crystallization process in acetone/ethanol system, and the single crystal sample was analyzed by single crystal diffraction. The obtained single crystal data were shown in Table 1, the molecular structure diagram of single crystal analysis was shown in FIG. 5, and the single crystal cell diagram was shown in FIG. 6.

TABLE 1
Single crystal data of the urea co-crystal A
Table 1. Crystal data and structure refinement for co-crystal A

| Empirical formula | C₂₇H₃₃N₉O₆ |
|---|---|
| Formula weight | 579.62 |
| Temperature/K | 293.15 |
| Crystal system | monoclinic |
| Space group | P2₁/c |
| a/Å | 12.8739(17) |
| b/Å | 9.4322(10) |
| c/Å | 25.691(2) |
| α/° | 90 |
| β/° | 99.871(10) |
| γ/° | 90 |
| Volume/Å³ | 3073.5(6) |
| Z | 4 |
| ρ_{calc}g/cm³ | 1.253 |
| μ/mm⁻¹ | 0.091 |
| F(000) | 1224.0 |
| Crystal size/mm³ | 0.35 × 0.3 × 0.25 |
| Radiation | MoKα(λ = 0.71073) |
| 2θ range for data collection/° | 5.774 to 52.744 |
| Index ranges | −16 ≤ h ≤ 15, −10 ≤ k ≤ 11, −29 ≤ |
| | l ≤ 32 |
| Reflections collected | 15245 |
| Independent reflections | 6274[Rᵢₙₜ = 0.0659, R_{sigma} = 0.1384] |
| Data/restraints/parameters | 6274/1/394 |
| Goodness-of-fit on F² | 1.038 |
| Final R indexes [I >= 2σ (I)] | R₁ = 0.1177, wR₂ = 0.3198 |
| Final R indexes [all data] | R₁ = 0.2541, wR₂ = 0.4131 |
| Largest diff. peak/hole/e Å⁻³ | 1.45/−0.30 |

### Test Example 1: Solubility Study Test on the Urea Co-Crystal A

In order to study the differences in solubilities between the urea co-crystal A prepared in Example 1 of the present invention and the pharmaceutical crystal form N−1 purchased from Srini Pharmaceuticals Pvt Ltd. The equilibrium solubilities (saturated solutions) of the urea co-crystal A prepared in Example 1 and the pharmaceutical crystal form N−1 were measured by external standard method in hydrochloric acid at pH=1.0 (0.1N), pure water and phosphate buffer solution at pH=6.8 at 25° C. and 37° C. in the present invention. The results were shown in Table 2 below.

TABLE 2
Solubility test

| Medium | Temperature | Crystal Form | Solubility |
|---|---|---|---|
| pH 1.0 | 25° C. | N-1 | 31.41 μg/ml |
| | | Co-crystal A | 48.62 μg/ml |
| | 37° C. | N-1 | 38.43 μg/ml |
| | | Co-crystal A | 57.33 μg/ml |
| pH 6.8 | 25° C. | N-1 | 22.09 μg/ml |
| | | Co-crystal A | 37.22 μg/ml |
| | 37° C. | N-1 | 29.36 μg/ml |
| | | Co-crystal A | 42.74 μg/ml |
| Pure water | 25° C. | N-1 | 36.66 μg/ml |
| | | Co-crystal A | 55.78 μg/ml |
| | 37° C. | N-1 | 49.25 μg/ml |
| | | Co-crystal A | 67.98 μg/ml |

The results of solubility test showed that the urea co-crystal A had significant advantages over pharmaceutical crystal form N−1 in terms of equilibrium solubilities at 25° C./37° C. in three media of pure water, pH 1.0 and pH 6.8. The solubilities of the urea co-crystal A in each medium and at each temperature was about 1.5 times of that of N−1, and the solubilities were thus significantly improved.

### Test Example 2: Stability Study Test on the Urea Co-Crystal A

In order to study the storage stability of the urea co-crystal A prepared in Example 1 of the present invention, the obtained sample was placed under high temperature and light irradiation to study the influence factors, and the sample was placed under 25±2° C. and 60±5% RH to perform the long-term stability test, and placed under 40° C.±2° C. and 75±5% RH to perform the accelerated stability test, and the results were shown in Table 3 below.

TABLE 3
Stability test

| Storage | Packaging | Storage | Crystalline | Purity |
|---|---|---|---|---|
| Condition | Mode | Time | Form Results | % |
| High | Inner bag of PE + | Start | Co-crystal A | 99.93 |
| Temperature | Outer aluminum | 1 month | Unchanged | 99.92 |
| (60° C.) | plastic bag | | | |
| Light Irradiation | Inner bag of PE + | Start | Co-crystal A | 99.93 |
| | Outer aluminum | 1 month | Unchanged | 99.93 |
| | plastic bag | | | |
| 25 ± 2° C. | Inner bag of PE + | Start | Co-crystal A | 99.93 |
| 60 ± 5% RH | Outer aluminum | 1 month | Unchanged | / |
| | plastic bag | 2 months | Unchanged | / |
| | | 3 months | Unchanged | / |
| | | 6 months | Unchanged | 99.92 |
| 40 ± 2° C. | Inner bag of PE + | Start | Co-crystal A | 99.93 |
| 75 ± 5% RH | Outer aluminum | 1 month | Unchanged | / |
| | plastic bag | 2 months | Unchanged | / |
| | | 3 months | Unchanged | / |
| | | 6 months | Unchanged | 99.92 |

The results of stability test showed that the urea co-crystal A had good crystalline form stability under all study conditions.

### Test Example 3: In Vivo Pharmacokinetic Test in Rat

1. Test Purpose

To study the plasma concentration level and pharmacokinetic characteristics of apixaban after a single oral administration of original pharmaceutical crystalline form N−1 of apixaban, the urea co-crystal A of apixaban and the urea co-crystal IV of apixaban from HEC Pharm Co., Ltd to rats.

2. Materials and Methods

2.1. Test Drugs

Apixaban pharmaceutical crystalline form N−1, provided by Srini Pharmaceuticals Pvt Ltd, nearly white solid, lot number Y20071, purity: 99.94%; The urea co-crystal A of apixaban, provided by the Crystalline Form Research Department of Chengdu Easton Biopharmaceuticals Co., Ltd, nearly white solid, purity: 99.93%.

The urea co-crystal IV of apixaban from HEC Pharm Co., Ltd, provided by the Crystalline Form Research Department of Chengdu Easton Biopharmaceuticals Co., Ltd, prepared according to Example 5 of patent CN106986868B, nearly white solid, purity: 99.93%.

2.2. Test Animals

18 SD rats with 9 male rats and 9 female rats, weighing 220-240 g, purchased by Chengdu Ensiweier Biotechnology Co., Ltd. from Hunan SJA Laboratory Animal Co., Ltd, License: SCXK (Xiang) 2019-0004.

2.3. Test Method

After the test drugs were prepared into 1.25 mg/kg uniform suspensions with corn oil, they were immediately administered orally to rats at 4 mL/kg, and 0.1 mL of blood was sampled from the jugular vein before the administration and 15 min, 30 min, 1 h, 2 h, 3 h, 4 h, 5 h, 6 h, 7 h, 8 h and 24 h after the administration, placed in EDTA-K2 tubes and centrifuged at 3000 r/min for 10 min. The plasma was separated and stored by freezing at −80° C. in refrigerator.

2.4. LC/MS/MS Biological Sample Analysis

50 μL of plasma was taken and evenly mixed with 5 μL of working solution or blank diluent, 150 μL of the internal-standard acetonitrile-containing precipitant was added thereto, which was shaken with vortex for 2 min, and centrifuged at 12000 r/min for 10 min. 2 μL of Supernatant was taken and mixed with 200 μL of pure water: acetonitrile (1:1), and then the resultant sample was injected with a volume of 3 μL for analysis.

2.5. Test Results

Apixaban pharmaceutical crystalline form N−1, the urea co-crystal A and the urea co-crystal IV from HEC Pharm Co., Ltd were tested using animal experiments, i.e. the average concentration (ng mL⁻¹) of API in the plasma of female and male rats were tested at different times after single oral administration, and the average drug concentration-time curves in plasma of female and male rats after single oral administration were plotted and shown in FIG. 9.1 and FIG. 9.2, and the main pharmacokinetic parameters thereof were shown in the table below:

TABLE 4
Main pharmacokinetic parameters of female
rats after single oral administration

| | | | The urea co-crystal |
|---|---|---|---|
| | Crystalline | The urea | IV from HEC Pharm |
| Parameters | form N-1 | co-crystal A | Co., Ltd |
| T_{1/2} (h) | 6.83 | 4.49 ± 0.10 | 6.55 ± 2.37 |
| Tₘₐₓ (h) | 5.83 ± 3.75 | 1.08 ± 0.72 | 1.00 ± 0.87 |
| Cₘₐₓ | 2570 ± 999 | 4653 ± 763 | 2563 ± 985 |
| (ng · mL⁻¹) | | | |
| AUCₗₐₛₜ | 27787 ± 13050 | 33636 ± 8113 | 23201 ± 8105 |
| (h · ng · mL⁻¹) | | | |
| Cl_F_obs | 0.56 | 0.30 ± 0.08 | 0.43 ± 0.15 |
| (mL/hr/kg) | | | |
| MRT (h) | 7.37 ± 1.79 | 5.55 ± 0.61 | 6.29 ± 0.87 |

TABLE 5
Main pharmacokinetic parameters of male
rats after single oral administration

| | | | The urea co-crystal |
|---|---|---|---|
| | Crystalline | The urea | IV from HEC Pharm |
| Parameters | form N-1 | co-crystal A | Co., Ltd |
| T_{1/2} (h) | 5.05 ± 1.43 | 5.53 ± 2.98 | 5.16 ± 1.14 |
| Tₘₐₓ (h) | 1.00 ± 0.87 | 1.00 ± 0.00 | 1.08 ± 0.88 |
| Cₘₐₓ | 4093 ± 3253 | 7970 ± 4475 | 4193 ± 862 |
| (ng · mL⁻¹) | | | |
| AUCₗₐₛₜ | 19764 ± 13974 | 25567 ± 14447 | 17052 ± 6085 |
| (h · ng · mL⁻¹) | | | |
| Cl_F_obs | 0.64 ± 0.32 | 0.45 ± 0.20 | 0.58 ± 0.27 |
| (mL/hr/kg) | | | |
| MRT (h) | 4.78 ± 0.75 | 4.16 ± 0.41 | 3.86 ± 0.07 |

Animal experiments showed that: 1. for female rats, the bioavailability of co-crystal A increased by 21% compared with the crystalline form N−1 of original research, and increased by 44.98% compared with the urea co-crystal IV from HEC Pharm Co., Ltd; 2. for male rats, the bioavailability of co-crystal A increased by 29.36% compared with the crystal form N−1 of original research, and increased by 49.94% compared with the urea co-crystal IV from HEC Pharm Co., Ltd. To summarize, it can be seen that the bioavailability of the urea co-crystal A obtained in the present invention was significantly improved compared with the pharmaceutical crystalline form N−1 and the urea co-crystal IV.

### Test Example 4: Dissolution Test of Preparations of the Urea Co-Crystal a and the Crystalline Form N−1

Formulation process: In accordance with the tablet formulation provided in Table 3 of DETAILED DESCRIPTION OF THE INVENTION in the original preparation patent CN109602713A, a tablet composition of apixaban of a specification of 5 mg was prepared and obtained by dry granulation method with the urea co-crystal A of apixaban as raw material.

Commercially available product: ELIQUIS from Bristol-Myers Squibb, 5 mg. The sample of the urea co-crystal A obtained in Example 1 was pressed via the preparation formulation process, and compared with the commercially available product to study the dissolution curves in media of pH 1.0 and pH 4.5. The data were shown in FIGS. 10.1 and 10.2, which showed that the dissolution behavior of the preparation product of the obtained urea co-crystal A was consistent with that of the commercially available product.

It can be seen from the above Test Examples that, compared with the pharmaceutical crystalline form N−1, the urea co-crystal A of apixaban provided by the present invention has the advantages of better dissolution performance, good crystalline form stability and physicochemical stability, significantly improved bioavailability, and the consistent dissolution effect with the commercially available product in various media.

It is obvious for the person of ordinary skill in the art that various modifications and variations can be made to the compound of the present application and the preparation method thereof without departing from the spirit or scope of the present application. Therefore, the protection scope of the present application covers every modification and variation made to the present application, provided that the modification or variation is within the scope covered by the claims and the equivalent embodiments thereof.

1. A urea co-crystal A of the compound shown in formula (I),
wherein a molar ratio of apixaban to urea in the co-crystal A is 1:2,

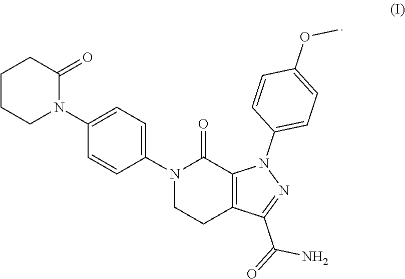 [SMILES: COc1ccc(-n2nc(C(N)=O)c3c2C(=O)N(c2ccc(N4CCCCC4=O)cc2)CC3)cc1]

2-15. (canceled)

16. The urea co-crystal A according to claim 1, wherein an X-ray powder diffraction pattern thereof has characteristic peaks at 2θ angles of 7.00±0.2°, 10.76±0.2°, 11.60±0.2°, 19.18±0.2°, 20.00±0.2°, 22.94±0.2°, 23.78±0.2° and 28.08±0.2°.

17. The urea co-crystal A according to claim 1, wherein the X-ray powder diffraction pattern thereof has characteristic peaks at 2θ angles of 7.00±0.2°, 10.76±0.2°, 11.60±0.2°, 12.52±0.2°, 19.18±0.2°, 20.00±0.2°, 22.94±0.2°, 23.78±0.2°, 25.16±0.2° and 28.08±0.2°.

18. The urea co-crystal A according to claim 1, wherein the X-ray powder diffraction pattern thereof has characteristic peaks at 2θ angles of 7.00±0.2°, 10.76±0.2°, 11.60±0.2°, 12.52±0.2°, 13.96±0.2°, 16.72±0.2°, 19.18±0.2°, 20.00±0.2°, 21.18±0.2°, 22.94±0.2°, 23.78±0.2°, 25.16±0.2°, 26.88±0.2°, 28.08±0.2° and 30.20±0.2°

19. The urea co-crystal A according to claim 1, wherein the urea co-crystal A has an X-ray powder diffraction pattern substantially shown in FIG. 1.

20. The urea co-crystal A according to claim 1, wherein a DSC thermogram thereof has an endothermic peak at 176±5° C.

21. The urea co-crystal A according to claim 20, wherein the DSC thermogram thereof is substantially shown in FIG. 2.

22. The urea co-crystal A according to claim 1, wherein a TGA diagram is substantially shown in FIG. 3.

23. The urea co-crystal A according to claim 1, wherein a NMR spectrum thereof is substantially shown in FIG. 4.

24. The urea co-crystal A according to claim 1, wherein the urea co-crystal A is in a form of a pharmaceutical composition optionally comprising a pharmaceutically acceptable excipient.

25. A preparation method of the urea co-crystal A according to claim 1, comprising:
(1) adding the compound apixaban shown in formula (I) and a certain equivalent amount of urea into ethanol or a mixed solvent of ethanol and other solvents, dissolving at reflux under elevated temperature, and then cooling down to room temperature for crystallization for 5-24 h, wherein said other solvents are selected from ketones and esters;
(2) filtrating by suction and collecting the obtained solid, drying the same to yield the urea co-crystal A of apixaban.

26. The preparation method of the urea co-crystal A according to claim 25, wherein a molar ratio of apixaban to urea in step (1) is 1:4 to 1:12.

27. The preparation method of the urea co-crystal A according to claim 26, wherein the molar ratio of apixaban to urea in step (1) is 1:6 to 1:10.

28. The preparation method of the urea co-crystal A according to claim 25, wherein a mass to volume ratio of apixaban to the solvent in step (1) is 1:10 to 1:30 (g/ml).

29. The preparation method of the urea co-crystal A according to claim 25, wherein said other solvents in step (1) are selected from acetone, butanone, ethyl acetate, methyl acetate or isopropyl acetate.

30. A method of preventing or treating a disease related to venous thrombosis, comprising administering to the subject in need thereof the urea co-crystal A according to claim 1.
